(19) United States
(12) Patent Application Publication
(10) Pub. No.: US 2009/0105142 A1
(43) Pub. Date: Apr. 23, 2009

(54) TREATMENT WITH KALLIKREIN INHIBITORS

(75) Inventor: RICHARD MOSCICKI, Newton, MA (US)

Correspondence Address: LOWRIE, LANDO & ANASTASI, LLP, ONE MAIN STREET, SUITE 1100, CAMBRIDGE, MA, 02142, US

(73) Assignee: GENZYME CORPORATION, CAMBRIDGE, MA (US)

(21) Appl. No.: 12/196,357

(22) Filed: Aug. 22, 2008

Related U.S. Application Data
(60) Provisional application No. 60/957,526, filed on Aug. 23, 2007.

(51) Int. Cl. A61K 38/16; A61P 19/06

(52) U.S. Cl. 514/12

(57) ABSTRACT

Methods, kits and compositions are disclosed that include a non-naturally occurring kallikrein inhibitor and an optional additional gout therapeutic for the treatment of gout, such as acute gout.

### CROSS-REFERENCE TO RELATED APPLICATIONS

This application claims priority to U.S. Application Ser. No. 60/957,526, filed on Aug. 23, 2007. The disclosure of the prior application is considered part of (and is incorporated by reference in) the disclosure of this application.

### TECHNICAL FIELD

The invention relates to the treatment of gout by the administration of an inhibitor of plasma kallikrein activity, particularly a non-naturally occurring kallikrein inhibitor.

### BACKGROUND

Gout is a well-characterized inflammatory arthritis characterized by hyperuricemia and the formation of uric acid crystals within the affected joint(s). Additionally, tophi (nodules formed by collections of uric acid crystals) are commonly observed. The course of gout typically involves alternating (largely) asymptomatic periods and acute gout episodes. Acute gout is characterized by severe, disabling pain and arthralgia, with swelling and redness at the affected joint(s).

Chronic gouty arthritis, characterized by chronic synovitis, cartilage loss, and bone erosion, may develop after a prolonged course of gout.

### SUMMARY

Disclosed herein are methods for the treatment of gout, also known as gouty arthritis, by administration of a non-naturally occurring plasma kallikrein (pKal) inhibitor, e.g., a Kunitz domain pKal inhibitor polypeptide.

In one aspect, the invention provides methods for the treatment of gout, such as acute gout, by administration of an effective amount of a non-naturally occurring pKal inhibitor.

In one embodiment, the treatment reduces pain associated with gout or acute gout.

In one embodiment, the treatment improves or stabilizes joint function (e.g., range of motion, grip strength, and the like).

In one embodiment, the treatment improves patient function (e.g., the ability of the patient to accomplish tasks of daily living). In another embodiment, the treatment stabilizes patient function (e.g., patient function does not decrease). Patient function can be measured by any of the available gout-related, arthritis-related, or general performance measures, such as the gout assessment questionnaire (GAQ), the health assessment questionnaire (HAQ), Katz index of activities of daily living (KIADL), or instrumental activities of daily living (IADL).

In one embodiment, the non-naturally occurring pKal inhibitor is administered in combination with an additional gout therapeutic. Additional gout therapeutics may be therapeutics used in the treatment of acute gout (e.g., non-steroidal anti-inflammatory drugs (NSAIDs), corticosteroids (e.g., prednisone), and/or analgesics), or phagocytosis inhibiting agents (e.g., colchicine), or chronically administered gout therapeutics such as uric acid lowering agents (e.g., xanthine oxidase inhibiting agents (e.g., allopurinol), uricosuric agents (e.g., probenecid), and/or uric acid metabolizing agents (e.g., uricase).

In one embodiment, the additional gout therapeutic is an agent used in the treatment of acute gout, such as an NSAID, a phagocytosis inhibitor, or a corticosteroid.

In one aspect, the invention provides kits for the treatment of gout. The kits include a non-naturally occurring inhibitor of pKal, and instructions for administering the inhibitor to a subject having gout (e.g., acute gout).

In one embodiment, the kit further includes instructions for administration of an additional therapeutic for the treatment of gout, and may optionally contain the additional therapeutic. In one embodiment, the instructions provide a dosing regimen, dosing schedule, and/or route of administration of the pKal inhibitor that differs from the dosing regimen, dosing schedule and/or route of administration for the inhibitor in the absence of the additional gout therapeutic.

In another aspect, provided herein are the use of a non-naturally occurring pKal inhibitor for the manufacture of a medicament for the treatment of gout. The medicament may optionally include an additional gout therapeutic. In one embodiment, the additional gout therapeutic is an agent used in the treatment of acute gout, such as an NSAID, a phagocytosis inhibitor, or a corticosteroid.

The non-naturally occurring kallikrein inhibitor used in any disclosed method, kit or composition can have one or more of the characteristics described below.

The kallikrein inhibitor can have a Ki for plasma kallikrein of less than 50 nM, 40 nM, 30 nM, 20 nM, 5 nM, 1 nM, 500 pM, 100 pM, 50 pM, e.g., about 44 pM. The pKal inhibitor can preferentially inhibit pKal at least 100, 200, 500, or 1000 more than another kallikrein, e.g., human urine kallikrein, or another protease, e.g., plasmin or thrombin.

In one embodiment, the kallikrein inhibitor includes a polypeptide that includes a Kunitz domain such as the amino acid sequence: Xaa1 Xaa2 Xaa3 Xaa4 Cys Xaa6 Xaa7 Xaa8 Xaa9 Xaa10 Xaa11 Gly Xaa13 Cys Xaa15 Xaa16 Xaa17 Xaa18 Xaa19 Xaa20 Xaa21 Xaa22 Xaa23 Xaa24 Xaa25 Xaa26 Xaa27 Xaa28 Xaa29 Cys Xaa31 Xaa32 Phe Xaa34 Xaa35 Gly Gly Cys Xaa39 Xaa40 Xaa41 Xaa42 Xaa43 Xaa44 Xaa45 Xaa46 Xaa47 Xaa48 Xaa49 Xaa50 Cys Xaa52 Xaa53 Xaa54 Cys Xaa56 Xaa57 Xaa58 (SEQ ID NO:1).

The framework of the Kunitz domain can be human or can differ from a human Kunitz domain framework by fewer than six, five, four, three, or two amino acids. For example, the framework of the Kunitz domain can be the framework of one of the Kunitz domains of human lipoprotein-associated coagulation inhibitor (LACI) protein, e.g., the first second or third Kunitz domain. LACI is also known as “Tissue Factor Pathway Inhibitor” or “TFPI”. Typically, the polypeptide differs from BPTI and/or one or more of the LACI Kunitz domains by at least one, two, three, or four amino acids, e.g., at least one, two or three amino acids in the binding loops and/or at least two, three, four, or six amino acids in the framework region. For example, the polypeptide can include a non-naturally occurring Kunitz domain that is derived from a naturally occurring Kunitz domain, e.g., a human Kunitz domain. In one embodiment, an inhibitor that includes a Kunitz domain binds to plasma kallikrein with an affinity that is at least 10, 100, or 500 fold better than BPTI and/or LACI.

In one embodiment, the polypeptide that inhibits kallikrein is not immunogenic on second use.

In one embodiment, the polypeptide that inhibits kallikrein can have one or more of the following features: Xaa1, Xaa2, Xaa3, Xaa4, Xaa56, Xaa57 or Xaa58 are each individually an amino acid or absent; Xaa10 is an amino acid selected from the group consisting of: Asp and Glu; Xaa11 is an amino acid selected from the group consisting of: Asp, Gly, Ser, Val, Asn, Ile, Ala and Thr; Xaa13 is an amino acid selected from the group consisting of: Arg, His, Pro, Asn, Ser, Thr, Ala, Gly, Lys and Gln; Xaa15 is an amino acid selected from the group consisting of: Arg, Lys, Ala, Ser, Gly, Met, Asn and Gln; Xaa16 is an amino acid selected from the group consisting of: Ala, Gly, Ser, Asp and Asn; Xaa17 is an amino acid selected from the group consisting of: Ala, Asn, Ser, Ile, Gly, Val, Gln and Thr; Xaa18 is an amino acid selected from the group consisting of: His, Leu, Gln and Ala; Xaa19 is an amino acid selected from the group consisting of: Pro, Gln, Leu, Asn and Ile; Xaa21 is an amino acid selected from the group consisting of: Trp, Phe, Tyr, His and Ile; Xaa22 is an amino acid selected from the group consisting of: Tyr and Phe; Xaa23 is an amino acid selected from the group consisting of: Tyr and Phe; Xaa31 is an amino acid selected from the group consisting of: Glu, Asp, Gln, Asn, Ser, Ala, Val, Leu, Ile and Thr; Xaa32 is an amino acid selected from the group consisting of: Glu, Gln, Asp Asn, Pro, Thr, Leu, Ser, Ala, Gly and Val; Xaa34 is an amino acid selected from the group consisting of: Thr, Ile, Ser, Val, Ala, Asn, Gly and Leu; Xaa35 is an amino acid selected from the group consisting of: Tyr, Trp and Phe; Xaa39 is an amino acid selected from the group consisting of: Glu, Gly, Ala, Ser and Asp; Xaa40 is an amino acid selected from the group consisting of: Gly and Ala; Xaa43 is an amino acid selected from the group consisting of: Asn and Gly; Xaa45 is an amino acid selected from the group consisting of: Phe and Tyr; and wherein the polypeptide inhibits kallikrein.

In a particular embodiment, individual amino acid positions of a kallikrein inhibitor that includes the amino acid sequence of SEQ ID NO:1 has one or more of the following: Xaa10 is Asp, Xaa11 is Asp, Xaa13 is Pro, Xaa15 is Arg, Xaa16 is Ala, Xaa17 is Ala, Xaa18 is His, Xaa19 is Pro, Xaa21 is Trp, Xaa31 is Glu, Xaa32 is Glu, Xaa34 is Ile, Xaa35 is Tyr, Xaa39 is Glu.

The polypeptide that inhibits kallikrein can include (or consist of) the following amino acid sequence: Met His Ser Phe Cys Ala Phe Lys Ala Asp Asp Gly Pro Cys Arg Ala Ala His Pro Arg Trp Phe Phe Asn Ile Phe Thr Arg Gln Cys Glu Glu Phe Ile Tyr Gly Gly Cys Glu Gly Asn Gln Asn Arg Phe Glu Ser Leu Glu Glu Cys Lys Lys Met Cys Thr Arg Asp (amino acids 3-60 of SEQ ID NO:2), or a fragment thereof, e.g., a fragment that binds and inhibits kallikrein. For example, the polypeptide can have fewer than 80, 70, 65, 60, 58, 55 or 52 amino acids.

The polypeptide that inhibits kallikrein can include (or consist of) a polypeptide described in U.S. Pat. No. 5,786,328, the contents of which are incorporated by reference.

Methods, kits and compositions described herein can include an inhibitor that comprises a non-naturally occurring Kunitz domain polypeptide having any of the amino acid sequences described herein and an additional flanking sequence of one to six amino acids at the amino and/or carboxy terminal end domains. Such additional amino acids may be artifacts of expressing a particular non-naturally occurring kallikrein inhibitor polypeptide or Kunitz domain polypeptide in any of a variety of recombinant expression vector systems, such as used in yeast, bacteria, mammalian cell lines, insect cells, and the like. Preferably, such additional amino acids at the amino and/or carboxy termini of a non-naturally occurring Kunitz domain described herein do not diminish the affinity for kallikrein or kallikrein inhibition activity of the domain or a polypeptide comprising the domain.

The inhibitor polypeptide can include a non-naturally occurring Kunitz domain polypeptide having an amino acid sequence of SEQ ID NO:1 and an amino terminal flanking sequence as the result of producing the polypeptide as a recombinant protein in yeast. An example of a particularly preferred yeast recombinant expression system comprises fusing a nucleotide coding sequence for a non-naturally occurring Kunitz domain of SEQ ID NO:1 to a nucleotide sequence encoding the matα Prepro peptide leader sequence of Saccharomyces cerevisiae and expressing the recombinant coding sequence in the yeast Pichia pastoris. The resulting expressed fusion protein comprises an amino acid sequence of SEQ ID NO:1 and an amino terminal flanking dipeptide, Glu-Ala. A particularly preferred species of an inhibitor polypeptide of the invention produced in a yeast expression system has the amino acid sequence of SEQ ID NO:2:

| | |
|---|---|
| Glu Ala Met His Ser Phe Cys Ala Phe Lys Ala Asp Asp Gly Pro Cys Arg Ala Ala | (SEQ ID NO: 2) |
| His Pro Arg Trp Phe Phe Asn Ile Phe Thr Arg Gln Cys Glu Glu Phe Ile Tyr Gly Gly Cys | |
| Glu Gly Asn Gln Asn Arg Phe Glu Ser Leu Glu Glu Cys Lys Lys Met Cys Thr Arg Asp. | |

In one embodiment, the polypeptide that inhibits pKal is modified, e.g., to include one or more moieties, e.g., one or more moieties that extend half life of the polypeptide, e.g., a polymer moiety or a plurality of polymer moieties, e.g., as described in U.S. Patent Publication No. 2005/0089515. For example, the polypeptide can include a plurality of polyethylene glycol moieties, e.g., one on an N-terminal amine and one attached to each lysine of the polypeptide. The polyethylene glycol moieties can be less than 10, 8, 7, or 6 kDa in average molecular weight. In other embodiments, the moiety can be, e.g., serum albumin, e.g., human serum albumin. Other exemplary modifications include a label, e.g., a radioactive or MRI-detectable label. In some embodiments, the polypeptide is part of a mixture that includes modified and unmodified polypeptides that inhibit kallikrein. For example, the mixture can include one or more modified polypeptides that inhibit kallikrein and that include a polymer moiety such as a polyethylene glycol moiety and one or more unmodified polypeptides that inhibit kallikrein and do not include a polymer moiety. In one embodiment, approximately 10%, 20%, 30%, 40%, 50%, 60%, 70%, 80%, 90% or all of the polypeptides that inhibit kallikrein in the mixture are modified.

The pKal inhibitor polypeptides useful in the methods, compositions and kits may be any of the non-naturally occurring Kunitz domain polypeptides described herein or larger polypeptides comprising any such Kunitz domains, provided the pKal inhibitor polypeptides bind and inhibit pKal as determined in standard assays.

The methods described herein include administering an effective amount of the non-naturally occurring pKal inhibitor. Such an amount can be an amount sufficient to produce an improvement detectable to one skilled in the art, to ameliorate at least one symptom, or to modulate (e.g., improve) at least one physiological parameter, e.g., to a statistically significant degree.

Preferred compositions, e.g., used in any method or kit described herein, may further comprise one or more pharmaceutically acceptable buffers, carriers, and excipients, which may provide a desirable feature to the composition including, but not limited to, enhanced administration of the composition to a patient, enhanced circulating half-life of the inhibitor, enhanced compatibility of the composition with patient blood chemistry, enhanced storage of the composition, and/or enhanced efficacy of the composition upon administration to a patient.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

FIG. 1 shows a portion of a DNA and corresponding deduced amino acid for an exemplary kallikrein inhibitor polypeptide in plasmid pPIC-K503. The inserted DNA encodes the matα Prepro signal peptide of Saccharomyces cerevisiae (underlined) fused in frame to the amino terminus of the PEP-1 polypeptide having the amino acid sequence enclosed by the boxed area. The amino acid sequence of the PEP-1 polypeptide shown in the boxed region is SEQ ID NO:2, and the corresponding nucleotide coding sequence is SEQ ID NO:3. The dashed arrows indicate the location and direction of two PCR primer sequences in AOX regions that were used to produce sequencing templates. DNA sequence for the entire nucleotide sequence of the figure includes the structural coding sequence for the fusion protein and is designated SEQ ID NO:27. The double underlined portion of the sequence indicates a diagnostic probe sequence. BstB I and EcoR I indicate locations of their respective palindromic, hexameric, restriction endonuclease sites in the sequence. Asterisks denote translational stop codons. See text for details.

FIGS. 2A and 2B show an alignment of exemplary amino acid sequences, the native LACI sequence from which these variants were derived (SEQ ID NO:32), and other known Kunitz domains (SEQ ID NOS:29-31 and 33-53). Cysteine residues are highlighted.

### DETAILED DESCRIPTION

The inventors present herein new methods for the treatment of gout, particularly acute gout, by the administration of a non-naturally occurring pKal inhibitor.

Kunitz Domain pKal Inhibitors

A number of useful inhibitors of pKal include a Kunitz domain.

As used herein, a “Kunitz domain” is a polypeptide domain having at least 51 amino acids and containing at least two, and preferably three, disulfides. The domain is folded such that the first and sixth cysteines, the second and fourth, and the third and fifth cysteines form disulfide bonds (e.g., in a Kunitz domain having 58 amino acids, cysteines can be present at positions corresponding to amino acids 5, 14, 30, 38, 51, and 55, according to the number of the BPTI homologous sequences provided below, and disulfides can form between the cysteines at position 5 and 55, 14 and 38, and 30 and 51), or, if two disulfides are present, they can form between a corresponding subset of cysteines thereof. The spacing between respective cysteines can be within 7, 5, 4, 3, 2, 1 or 0 amino acids of the following spacing between positions corresponding to: 5 to 55, 14 to 38, and 30 to 51, according to the numbering of the BPTI sequence provided below. The BPTI sequence can be used as a reference to refer to specific positions in any generic Kunitz domain. Comparison of a Kunitz domain of interest to BPTI can be performed by identifying the best fit alignment in which the number of aligned cysteines in maximized.

The 3D structure (at high resolution) of the Kunitz domain of BPTI is known. One of the X-ray structures is deposited in the Brookhaven Protein Data Bank as “6PTI”. The 3D structure of some BPTI homologues (Eigenbrot et al., (1990) Protein Engineering, 3(7):591-598; Hynes et al., (1990) Biochemistry, 29:10018-10022) are known. At least eighty one Kunitz domain sequences are known. Known human homologues include three Kunitz domains of LACI (Wun et al., (1988) J. Biol. Chem. 263(13):6001-6004; Girard et al., (1989) Nature, 338:518-20; Novotny et al, (1989) J. Biol. Chem., 264(31):18832-18837) two Kunitz domains of Inter-α-Trypsin Inhibitor, APP-I (Kido et al., (1988) J. Biol. Chem., 263(34):18104-18107), a Kunitz domain from collagen, three Kunitz domains of TFPI-2 (Sprecher et al., (1994) PNAS USA, 91:3353-3357), the Kunitz domains of hepatocyte growth factor activator inhibitor type 1, the Kunitz domains of Hepatocyte growth factor activator inhibitor type 2, the Kunitz domains described in U.S. Patent Publication No.: 20040152633. LACI is a human serum phosphoglycoprotein with a molecular weight of 39 kDa (amino acid sequence in Table 1) containing three Kunitz domains.

TABLE 1
Exemplary Natural Kunitz Domains

| | | |
|---|---|---|
| LACI: | 1 | MIYTMKKVHA LWASVCLLLN LAPAPLNAds eedeehtiit dtelpplklM |
| (SEQ ID | 51 | HSFCAFKADD GPCKAIMKRF FFNIFTRQCE EFIYGGCEGN QNRFESLEEC |
| NO. 54) | 101 | KKMCTRDnan riikttlqqe kpdfCfleed pgiCrgyitr yfynnqtkqC |
| | 151 | erfkyggClg nmnnfetlee CkniCedgpn gfqvdnygtq lnavnnsltp |
| | 201 | qstkvpslfe fhgpswCltp adrglCrane nrfyynsvig kCrpfkysgC |
| | 251 | ggnennftsk qeClraCkkg fiqriskggl iktkrkrkkq rvkiayeeif |
| | 301 | vknm |

| | |
|---|---|
| | The signal sequence (1-28) is uppercase and underscored |
| | LACI-K1 (50-107) is uppercase |
| | LACI-K2 (121-178) is underscored |
| | LACI-K3 (211-270) is bold |
| BPTI | 1 2 3 4 5 |
| (SEQ ID | 1234567890123456789012345678901234567890123456789012345678 |
| NO: 55) | RPDFCLEPPYTGPCKARIIRYFYNAKAGLCQTFVYGGCRAKRNNFKSAEDCMRTCGGA |

The Kunitz domains above are referred to as LACI-K1 (residues 50 to 107), LACI-K2 (residues 121 to 178), and LACI-K3 (213 to 270). The cDNA sequence of LACI is reported in Wun et al. (J. Biol. Chem., 1988, 263(13):6001-6004). Girard et al. (Nature, 1989, 338:518-20) reports mutational studies in which the PI residues of each of the three Kunitz domains were altered. LACI-K1 inhibits Factor VIIa (F.VIIa) when F.VIIa is complexed to tissue factor and LACI-K2 inhibits Factor Xa.

Proteins containing exemplary Kunitz domains include the following, with SWISS-PROT Accession Numbers in parentheses:

A4_HUMAN (P05067), A4_MACFA (P53601), A4_MACMU (P29216),
A4_MOUSE (P12023), A4_RAT (P08592), A4_SAISC (Q95241),
AMBP_PLEPL (P36992), APP2_HUMAN (Q06481), APP2_RAT (P15943),
AXP1_ANTAF (P81547), AXP2_ANTAF (P81548), BPT1_BOVIN (P00974),
BPT2_BOVIN (P04815), CA17_HUMAN (Q02388), CA36_CHICK (P15989),
CA36_HUMAN (P12111), CRPT_BOOMI (P81162), ELAC_MACEU (O62845),
ELAC_TRIVU (Q29143), EPPI_HUMAN (O95925), EPPI_MOUSE (Q9DA01),
HTIB_MANSE (P26227), IBP_CARCR (P00993), IBPC_BOVIN (P00976),
IBPI_TACTR (P16044), IBPS_BOVIN (P00975), ICS3_BOMMO (P07481),
IMAP_DROFU (P11424), IP52_ANESU (P10280), ISC1_BOMMO (P10831),
ISC2_BOMMO (P10832), ISH1_STOHE (P31713), ISH2_STOHE (P81129),
ISIK_HELPO (P00994), ISP2_GALME (P81906), IVB1_BUNFA (P25660),
IVB1_BUNMU (P00987), IVB1_VIPAA (P00991), IVB2_BUNMU (P00989),
IVB2_DABRU (P00990), IVB2_HEMHA (P00985), IVB2_NAJNI (P00986),
IVB3_VIPAA (P00992), IVBB_DENPO (P00983), IVBC_NAJNA (P19859),
IVBC_OPHHA (P82966), IVBE_DENPO (P00984), IVBI_DENAN (P00980),
IVBI_DENPO (P00979), IVBK_DENAN (P00982), IVBK_DENPO (P00981),
IVBT_ERIMA (P24541), IVBT_NAJNA (P20229), MCPI_MELCP (P82968),
SBPI_SARBU (P26228), SPT3_HUMAN (P49223), TKD1_BOVIN (Q28201),
TKD1_SHEEP (Q29428), TXCA_DENAN (P81658), UPTI_PIG (Q29100),
AMBP_BOVIN (P00978), AMBP_HUMAN (P02760), AMBP_MERUN (Q62577),
AMBP_MESAU (Q60559), AMBP_MOUSE (Q07456), AMBP_PIG (P04366),
AMBP_RAT (Q64240), IATR_HORSE (P04365), IATR_SHEEP (P13371),
SPT1_HUMAN (O43278), SPT1_MOUSE (Q9R097), SPT2_HUMAN (O43291),
SPT2_MOUSE (Q9WU03), TFP2_HUMAN (P48307), TFP2_MOUSE (O35536),
TFPI_HUMAN (P10646), TFPI_MACMU (Q28864), TFPI_MOUSE (O54819),
TFPI_RABIT (P19761), TFPI_RAT (Q02445), YN81_CAEEL (Q03610)

A variety of methods can be used to identify a Kunitz domain from a sequence database. For example, a known amino acid sequence of a Kunitz domain, a consensus sequence, or a motif (e.g., the ProSite Motif) can be searched against the GenBank sequence databases (National Center for Biotechnology Information, National Institutes of Health, Bethesda Md.), e.g., using BLAST; against Pfam database of HMMs (Hidden Markov Models) (e.g., using default parameters for Pfam searching; against the SMART database; or against the Propom database. For example, the Pfam Accession Number PF00014 of Pfam Release 9 provides numerous Kunitz domains and an HMM for identify Kunitz domains. A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314. The SMART database (Simple Modular Architecture Research Tool, EMBL, Heidelberg, Del.) of HMMs as described in Schultz et al. (1998), Proc. Natl. Acad. Sci. USA 95:5857 and Schultz et al. (2000) Nucl. Acids Res 28:231. The SMART database contains domains identified by profiling with the hidden Markov models of the HMMer2 search program (R. Durbin et al. (1998) Biological sequence analysis: probabilistic models of proteins and nucleic acids. Cambridge University Press). The database also is annotated and monitored. The Propom protein domain database consists of an automatic compilation of homologous domains (Corpet et al. (1999), Nucl. Acids Res. 27:263-267). Current versions of Propom are built using recursive PSI-BLAST searches (Altschul et al. (1997) Nucleic Acids Res. 25:3389-3402; Gouzy et al. (1999) Computers and Chemistry 23:333-340.) of the SWISS-PROT 38 and TREMBL protein databases. The database automatically generates a consensus sequence for each domain. Prosite lists the Kunitz domain as a motif and identifies proteins that include a Kunitz domain. See, e.g., Falquet et al. Nucleic Acids Res. 30:235-238 (2002).

Kunitz domains interact with target protease using, primarily, amino acids in two loop regions (“binding loops”). The first loop region is between about residues corresponding to amino acids 13-20 of BPTI. The second loop region is between about residues corresponding to amino acids 31-39 of BPTI. An exemplary library of Kunitz domains varies one or more amino acid positions in the first and/or second loop regions. Particularly useful positions to vary, when screening for Kunitz domains that interact with kallikrein or when selecting for improved affinity variants, include: positions 13, 15, 16, 17, 18, 19, 31, 32, 34, and 39 with respect to the sequence of BPTI. At least some of these positions are expected to be in close contact with the target protease. It is also useful to vary other positions, e.g., positions that are adjacent to the aforementioned positions in the three-dimensional structure.

The “framework region” of a Kunitz domain is defined as those residues that are a part of the Kunitz domain, but specifically excluding residues in the first and second binding loops regions, i.e., about residues corresponding to amino acids 13-20 of BPTI and 31-39 of BPTI. Conversely, residues that are not in the binding loop may tolerate a wider range of amino acid substitution (e.g., conservative and/or non-conservative substitutions).

In one embodiment, these Kunitz domains are variant forms of the looped structure including Kunitz domain 1 of human lipoprotein-associated coagulation inhibitor (LACI) protein. LACI contains three internal, well-defined, peptide loop structures that are paradigm Kunitz domains (Girard, T. et al., 1989. Nature, 338:518-520). Variants of Kunitz domain 1 of LACI described herein have been screened, isolated and bind kallikrein with enhanced affinity and specificity (see, for example, U.S. Pat. Nos. 5,795,865 and 6,057,287, incorporated herein by reference). These methods can also be applied to other Kunitz domain frameworks to obtain other Kunitz domains that interact with kallikrein, e.g., plasma kallikrein. Useful modulators of kallikrein function typically bind and/or inhibit kallikrein, as determined using kallikrein binding and inhibition assays.

An exemplary polypeptide that includes a Kunitz domain that inhibits kallikrein has the amino acid sequence defined by amino acids 3-60 of SEQ ID NO:2.

An exemplary polypeptide includes the amino acid sequence:

| | |
|---|---|
| Xaa1 Xaa2 Xaa3 Xaa4 Cys Xaa6 Xaa7 Xaa8 Xaa9 Xaa10 Xaa11 Gly Xaa13 Cys | (SEQ ID NO: 1) |
| Xaa15 Xaa16 Xaa17 Xaa18 Xaa19 Xaa20 Xaa21 Xaa22 Xaa23 Xaa24 Xaa25 Xaa26 | |
| Xaa27 Xaa28 Xaa29 Cys Xaa31 Xaa32 Phe Xaa34 Xaa35 Gly Gly Cys Xaa39 Xaa40 | |
| Xaa41 Xaa42 Xaa43 Xaa44 Xaa45 Xaa46 Xaa47 Xaa48 Xaa49 Xaa50 Cys Xaa52 Xaa53 | |
| Xaa54 Cys Xaa56 Xaa57 Xaa58 | |

“Xaa” refers to a position in a peptide chain that can be any of a number of different amino acids. In a first example, Xaa can by any amino acid except cysteine. In another example, one or more of the following apply: Xaa10 can be Asp or Glu; Xaa11 can be Asp, Gly, Ser, Val, Asn, Ile, Ala or Thr; Xaa13 can be Pro, Arg, His, Asn, Ser, Thr, Ala, Gly, Lys or Gln; Xaa15 can be Arg, Lys, Ala, Ser, Gly, Met, Asn or Gln; Xaa16 can be Ala, Gly, Ser, Asp or Asn; Xaa17 can be Ala, Asn, Ser, Ile, Gly, Val, Gln or Thr; Xaa18 can be His, Leu, Gln or Ala; Xaa19 can be Pro, Gln, Leu, Asn or Ile; Xaa21 can be Trp, Phe, Tyr, His or Ile; Xaa31 can be Glu, Asp, Gln, Asn, Ser, Ala, Val, Leu, Ile or Thr; Xaa32 can be Glu, Gln, Asp Asn, Pro, Thr, Leu, Ser, Ala, Gly or Val; Xaa34 can be Ile, Thr, Ser, Val, Ala, Asn, Gly or Leu; Xaa35 can be Tyr, Trp or Phe; Xaa39 can be Glu, Gly, Ala, Ser or Asp. Amino acids Xaa6, Xaa7, Xaa8, Xaa9, Xaa20, Xaa24, Xaa25, Xaa26, Xaa27, Xaa28, Xaa29, Xaa41, Xaa42, Xaa44, Xaa46, Xaa47, Xaa48, Xaa49, Xaa50, Xaa52, Xaa53 and Xaa54 can be any amino acid.

Additionally, each of the first four and at last three amino acids of SEQ ID NO:1 can optionally be present or absent and can be any amino acid, if present, e.g., any non-cysteine amino acid.

In one embodiment, the polypeptide has a sequence with one or more of the following properties: Xaa11 can be Asp, Gly, Ser or Val; Xaa13 can be Pro, Arg, His or Asn; Xaa15 can be Arg or Lys; Xaa16 can be Ala or Gly; Xaa17 can be Ala, Asn, Ser or Ile; Xaa18 can be His, Leu or Gln; Xaa19 can be Pro, Gln or Leu; Xaa21 can be Trp or Phe; Xaa31 is Glu; Xaa32 can be Glu or Gln; Xaa34 can be Ile, Thr or Ser; Xaa35 is Tyr; and Xaa39 can be Glu, Gly or Ala.

An exemplary polypeptide can include the following amino acids: Xaa10 is Asp; Xaa 11 is Asp; Xaa13 can be Pro or Arg; Xaa15 is Arg; Xaa16 can be Ala or Gly; Xaa17 is Ala; Xaa18 is His; Xaa19 is Pro; Xaa21 is Trp; Xaa31 is Glu; Xaa32 is Glu; Xaa34 can be Ile or Ser; Xaa35 is Tyr; and Xaa39 is Gly.

It is also possible to use portions of the polypeptides described herein. For example, polypeptides could include binding domains for specific kallikrein epitopes. For example, the binding loops of Kunitz domains can by cyclized and used in isolation or can be grafted onto another domain, e.g., a framework of another Kunitz domain. It is also possible to remove one, two, three, or four amino acids from the N-terminus of an amino acid sequence described herein, and/or one, two, three, four, or five amino acids from the C-terminus of an amino acid sequence described herein.

Examples of sequences encompassed by SEQ ID NO:1 are described by the following (where not indicated, “Xaa” refers to any amino acid, any non-cysteine amino acid or any amino acid from the same set of amino acids that are allowed for SEQ ID NO:1):

```
(SEQ ID NO: 33)
            Met His Ser Phe Cys Ala Phe Lys Ala Xaa10 Xaa11 Gly Xaa13 Cys Xaa15
Xaa16 Xaa17 Xaa18 Xaa19 Arg Xaa21 Phe Phe Asn Ile Phe Thr Arg Gln Cys Xaa31
Xaa32 Phe Xaa34 Xaa35 Gly Gly Cys Xaa39 Gly Asn Gln Asn Arg Phe Glu Ser Leu Glu
Glu Cys Lys Lys Met Cys Thr Arg Asp.
(amino acids 3-60 of SEQ ID NO: 2)
            Met His Ser Phe Cys Ala Phe Lys Ala Asp Asp Gly Pro Cys Arg Ala Ala His Pro
Arg Trp Phe Phe Asn Ile Phe Thr Arg Gln Cys Glu Glu Phe Ile Tyr Gly Gly Cys Glu Gly
Asn Gln Asn Arg Phe Glu Ser Leu Glu Glu Cys Lys Lys Met Cys Thr Arg Asp,
(SEQ ID NO: 4)
            Met His Ser Phe Cys Ala Phe Lys Ala Asp Asp Gly Pro Cys Lys Ala Asn His Leu
Arg Phe Phe Phe Asn Ile Phe Thr Arg Gln Cys Glu Glu Phe Ser Tyr Gly Gly Cys Gly
Gly Asn Gln Asn Arg Phe Glu Ser Leu Glu Glu Cys Lys Lys Met Cys Thr Arg Asp,
(SEQ ID NO: 5)
            Met His Ser Phe Cys Ala Phe Lys Ala Asp Asp Gly His Cys Lys Ala Asn His Gln
Arg Phe Phe Phe Asn Ile Phe Thr Arg Gln Cys Glu Glu Phe Thr Tyr Gly Gly Cys Gly
Gly Asn Gln Asn Arg Phe Glu Ser Leu Glu Glu Cys Lys Lys Met Cys Thr Arg Asp,
(SEQ ID NO: 6)
            Met His Ser Phe Cys Ala Phe Lys Ala Asp Asp Gly His Cys Lys Ala Asn His Gln
Arg Phe Phe Phe Asn Ile Phe Thr Arg Gln Cys Glu Gln Phe Thr Tyr Gly Gly Cys Ala
Gly Asn Gln Asn Arg Phe Glu Ser Leu Glu Glu Cys Lys Lys Met Cys Thr Arg Asp,
(SEQ ID NO: 7)
            Met His Ser Phe Cys Ala Phe Lys Ala Asp Asp Gly His Cys Lys Ala Ser Leu Pro
Arg Phe Phe Phe Asn Ile Phe Thr Arg Gln Cys Glu Glu Phe Ile Tyr Gly Gly Cys Gly Gly
Asn Gln Asn Arg Phe Glu Ser Leu Glu Glu Cys Lys Lys Met Cys Thr Arg Asp,
(SEQ ID NO: 8)
            Met His Ser Phe Cys Ala Phe Lys Ala Asp Asp Gly His Cys Lys Ala Asn His Gln
Arg Phe Phe Phe Asn Ile Phe Thr Arg Gln Cys Glu Glu Phe Ser Tyr Gly Gly Cys Gly
Gly Asn Gln Asn Arg Phe Glu Ser Leu Glu Glu Cys Lys Lys Met Cys Thr Arg Asp,
(SEQ ID NO: 9)
            Met His Ser Phe Cys Ala Phe Lys Ala Asp Asp Gly His Cys Lys Gly Ala His Leu
Arg Phe Phe Phe Asn Ile Phe Thr Arg Gln Cys Glu Glu Phe Ile Tyr Gly Gly Cys Glu Gly
Asn Gln Asn Arg Phe Glu Ser Leu Glu Glu Cys Lys Lys Met Cys Thr Arg Asp,
(SEQ ID NO: 10)
            Met His Ser Phe Cys Ala Phe Lys Ala Asp Asp Gly Arg Cys Lys Gly Ala His Leu
Arg Phe Phe Phe Asn Ile Phe Thr Arg Gln Cys Glu Glu Phe Ile Tyr Gly Gly Cys Glu Gly
Asn Gln Asn Arg Phe Glu Ser Leu Glu Glu Cys Lys Lys Met Cys Thr Arg Asp,
(SEQ ID NO: 11)
            Met His Ser Phe Cys Ala Phe Lys Ala Asp Gly Gly Arg Cys Arg Gly Ala His Pro
Arg Trp Phe Phe Asn Ile Phe Thr Arg Gln Cys Glu Glu Phe Ser Tyr Gly Gly Cys Gly
Gly Asn Gln Asn Arg Phe Glu Ser Leu Glu Glu Cys Lys Lys Met Cys Thr Arg Asp,
(SEQ ID NO: 12)
            Met His Ser Phe Cys Ala Phe Lys Ala Asp Asp Gly Pro Cys Arg Ala Ala His Pro
Arg Trp Phe Phe Asn Ile Phe Thr Arg Gln Cys Glu Glu Phe Ser Tyr Gly Gly Cys Gly
Gly Asn Gln Asn Arg Phe Glu Ser Leu Glu Glu Cys Lys Lys Met Cys Thr Arg Asp,
(SEQ ID NO: 13)
            Met His Ser Phe Cys Ala Phe Lys Ala Asp Val Gly Arg Cys Arg Gly Ala His Pro
Arg Trp Phe Phe Asn Ile Phe Thr Arg Gln Cys Glu Glu Phe Ser Tyr Gly Gly Cys Gly
Gly Asn Gln Asn Arg Phe Glu Ser Leu Glu Glu Cys Lys Lys Met Cys Thr Arg Asp,
(SEQ ID NO: 14)
            Met His Ser Phe Cys Ala Phe Lys Ala Asp Val Gly Arg Cys Arg Gly Ala Gln Pro
Arg Phe Phe Phe Asn Ile Phe Thr Arg Gln Cys Glu Glu Phe Ser Tyr Gly Gly Cys Gly
Gly Asn Gln Asn Arg Phe Glu Ser Leu Glu Glu Cys Lys Lys Met Cys Thr Arg Asp,
(SEQ ID NO: 15)
            Met His Ser Phe Cys Ala Phe Lys Ala Asp Asp Gly Ser Cys Arg Ala Ala His Leu
Arg Trp Phe Phe Asn Ile Phe Thr Arg Gln Cys Glu Glu Phe Ser Tyr Gly Gly Cys Gly
Gly Asn Gln Asn Arg Phe Glu Ser Leu Glu Glu Cys Lys Lys Met Cys Thr Arg Asp,
(SEQ ID NO: 16)
            Met His Ser Phe Cys Ala Phe Lys Ala Glu Gly Gly Ser Cys Arg Ala Ala His Gln
Arg Trp Phe Phe Asn Ile Phe Thr Arg Gln Cys Glu Glu Phe Ser Tyr Gly Gly Cys Gly
Gly Asn Gln Asn Arg Phe Glu Ser Leu Glu Glu Cys Lys Lys Met Cys Thr Arg Asp,
(SEQ ID NO: 17)
            Met His Ser Phe Cys Ala Phe Lys Ala Asp Asp Gly Pro Cys Arg Gly Ala His Leu
Arg Phe Phe Phe Asn Ile Phe Thr Arg Gln Cys Glu Glu Phe Ser Tyr Gly Gly Cys Gly
Gly Asn Gln Asn Arg Phe Glu Ser Leu Glu Glu Cys Lys Lys Met Cys Thr Arg Asp,
(SEQ ID NO: 18)
            Met His Ser Phe Cys Ala Phe Lys Ala Asp Asp Gly His Cys Arg Gly Ala Leu Pro
Arg Trp Phe Phe Asn Ile Phe Thr Arg Gln Cys Glu Glu Phe Ser Tyr Gly Gly Cys Gly
Gly Asn Gln Asn Arg Phe Glu Ser Leu Glu Glu Cys Lys Lys Met Cys Thr Arg Asp,
(SEQ ID NO: 19)
            Met His Ser Phe Cys Ala Phe Lys Ala Asp Ser Gly Asn Cys Arg Gly Asn Leu Pro
Arg Phe Phe Phe Asn Ile Phe Thr Arg Gln Cys Glu Glu Phe Ser Tyr Gly Gly Cys Gly
Gly Asn Gln Asn Arg Phe Glu Ser Leu Glu Glu Cys Lys Lys Met Cys Thr Arg Asp,
(SEQ ID NO: 20)
            Met His Ser Phe Cys Ala Phe Lys Ala Asp Ser Gly Arg Cys Arg Gly Asn His Gln
Arg Phe Phe Phe Asn Ile Phe Thr Arg Gln Cys Glu Glu Phe Ser Tyr Gly Gly Cys Gly
Gly Asn Gln Asn Arg Phe Glu Ser Leu Glu Glu Cys Lys Lys Met Cys Thr Arg Asp,
(SEQ ID NO: 21)
            Met His Ser Phe Cys Ala Phe Lys Ala Asp Gly Gly Arg Cys Arg Ala Ile Gln Pro
Arg Trp Phe Phe Asn Ile Phe Thr Arg Gln Cys Glu Glu Phe Ser Tyr Gly Gly Cys Gly
Gly Asn Gln Asn Arg Phe Glu Ser Leu Glu Glu Cys Lys Lys Met Cys Thr Arg Asp,
(SEQ ID NO: 22)
            Met His Ser Phe Cys Ala Phe Lys Ala Asp Asp Gly Arg Cys Arg Gly Ala His Pro
Arg Trp Phe Phe Asn Ile Phe Thr Arg Gln Cys Glu Glu Phe Ser Tyr Gly Gly Cys Gly
Gly Asn Gln Asn Arg Phe Glu Ser Leu Glu Glu Cys Lys Lys Met Cys Thr Arg Asp.
```

Additional examples of sequence include those that differ by at least one amino acid, but fewer than seven, six, five, four, three, or two amino acids differences relative to an amino acid sequence described herein, e.g., an amino acid sequence provided above. In one embodiment, fewer than three, two, or one differences are in one of the binding loops. For example, the first binding loop may have no differences relative to an amino acid sequence described herein, e.g., an amino acid sequence provided above. In another example, neither the first nor the second binding loop differs from an amino acid sequence described herein, e.g., an amino acid sequence provided above.

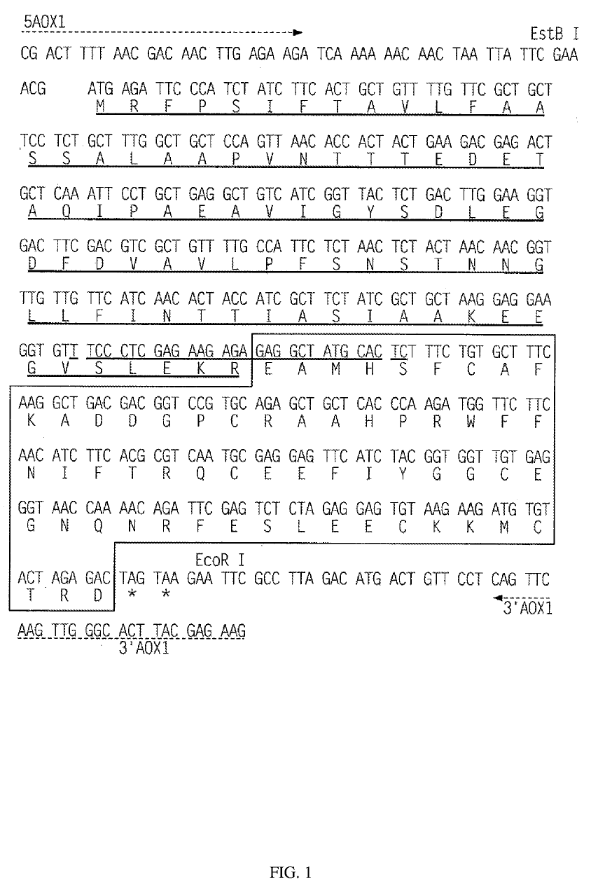

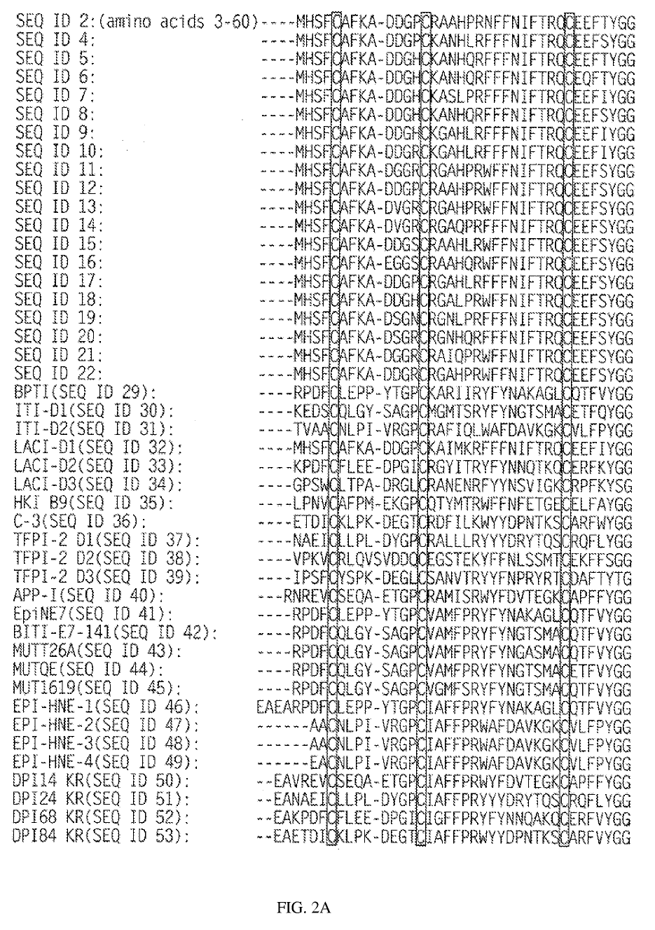

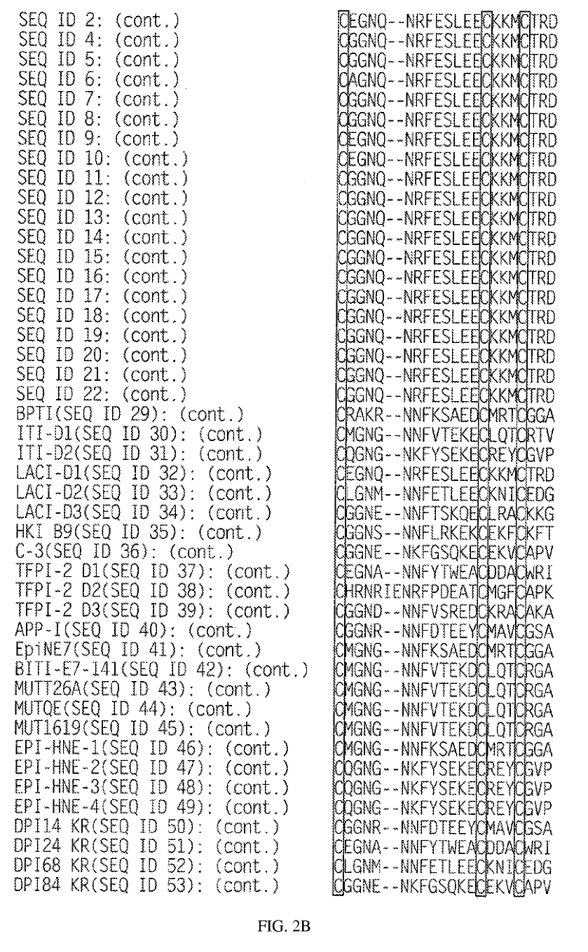

FIGS. 2A and 2B provide an amino acid sequence alignment of these sequences, the native LACI sequence from which these variants were derived (SEQ ID NO:32), and other known Kunitz domains (SEQ ID NOS: 29-31 and 33-53). Still others polypeptides that inhibit kallikrein include an about 58-amino acid sequence of amino acids 3-60 of SEQ ID NO:2 or the PEP-1 polypeptide having the 60-amino acid sequence of SEQ ID NO:2. The term “PEP-1” and “DX-88” as used herein refer to the 60-amino acid sequence of SEQ ID NO:2. A nucleotide sequence encoding the amino acid sequence of SEQ ID NO:2 is provided in SEQ ID NO:3 (see, e.g., nucleotides 309-488 in FIG. 1). It is understood that based on the known genetic code, degenerate forms of the nucleotide sequence of SEQ ID NO:3 can be obtained by simply substituting one or more of the known degenerate codons for each amino acid encoded by the nucleotide sequence. Nucleotides 7-180 of SEQ ID NO:3, and degenerate forms thereof, encode the non-naturally occurring Kunitz domain polypeptide that includes the 58-amino acid sequence of amino acids 3-60 of SEQ ID NO:2, a related sequence, or a functional fragment thereof.

In one embodiment, the polypeptide is other than aprotinin, e.g., differs from aprotinin, by at least one, two, three, five, ten, or fifteen amino acids.

Polypeptides described herein can be made synthetically using any standard polypeptide synthesis protocol and equipment. For example, the stepwise synthesis of a polypeptide can be carried out by the removal of an amino (N) terminal-protecting group from an initial (i.e., carboxy-terminal) amino acid, and coupling thereto of the carboxyl end of the next amino acid in the sequence of the polypeptide. This amino acid is also suitably protected. The carboxyl group of the incoming amino acid can be activated to react with the N-terminus of the bound amino acid by formation into a reactive group such as formation into a carbodiimide, a symmetric acid anhydride, or an “active ester” group such as hydroxybenzotriazole or pentafluorophenyl esters. Preferred solid-phase peptide synthesis methods include the BOC method, which utilizes tert-butyloxycarbonyl as the I-amino protecting group, and the FMOC method, which utilizes 9-fluorenylmethloxycarbonyl to protect the alpha-amino of the amino acid residues. Both methods are well known to those of skill in the art (Stewart, J. and Young, J., Solid-Phase Peptide Synthesis (W. H. Freeman Co., San Francisco 1989); Merrifield, J., 1963. Am. Chem. Soc., 85:2149-2154; Bodanszky, M. and Bodanszky, A., The Practice of Peptide Synthesis (Springer-Verlag, New York 1984)). If desired, additional amino- and/or carboxy-terminal amino acids can be designed into the amino acid sequence and added during polypeptide synthesis.

Polypeptides can also be produced using recombinant technology. Recombinant methods can employ any of a number of cells and corresponding expression vectors, including but not limited to bacterial expression vectors, yeast expression vectors, baculovirus expression vectors, mammalian viral expression vectors, and the like. A polypeptide described herein can be produced by a transgenic animal, e.g., in the mammary gland of a transgenic animal. In some cases, it could be necessary or advantageous to fuse the coding sequence for a polypeptide that inhibits kallikrein (e.g., a polypeptide that includes a Kunitz domain) to another coding sequence in an expression vector to form a fusion polypeptide that is readily expressed in a host cell. Part or all of the additional sequence can be removed, e.g., by protease digestion.

An exemplary recombinant expression system for producing a polypeptide that inhibits kallikrein (e.g., a polypeptide that includes a Kunitz domain) is a yeast expression vector, which permits a nucleic acid sequence encoding the amino acid sequence for the inhibitor polypeptide to be linked in the same reading frame with a nucleotide sequence encoding the MATα prepro leader peptide sequence of Saccharomyces cerevisiae, which in turn is under the control of an operable yeast promoter. The resulting recombinant yeast expression plasmid can be transformed by standard methods into the cells of an appropriate, compatible yeast host, which cells are able to express the recombinant protein from the recombinant yeast expression vector. Preferably, a host yeast cell transformed with such a recombinant expression vector is also able to process the fusion protein to provide an active inhibitor polypeptide. An other exemplary yeast host for producing recombinant polypeptides is Pichia pastoris.

As noted above, polypeptides that inhibit kallikrein can include a Kunitz domain polypeptide described herein. Some polypeptides can include an additional flanking sequence, preferably of one to six amino acids in length, at the amino and/or carboxy-terminal end, provided such additional amino acids do not significantly diminish kallikrein binding affinity or kallikrein inhibition activity so as to preclude use in the methods and compositions described herein. Such additional amino acids can be deliberately added to express a polypeptide in a particular recombinant host cell or can be added to provide an additional function, e.g., to provide a linker to another molecule or to provide an affinity moiety that facilitates purification of the polypeptide. Preferably, the additional amino acid(s) do not include cysteine, which could interfere with the disulfide bonds of the Kunitz domain.

An exemplary Kunitz domain polypeptide includes the amino acid sequence of residues 3-60 of SEQ ID NO:2. When expressed and processed in a yeast fusion protein expression system (e.g., based on the integrating expression plasmid pHIL-D2), such a Kunitz domain polypeptide retains an additional amino terminal Glu-Ala dipeptide from the fusion with the MATalpha-prepro leader peptide sequence of S. cerevisiae. When secreted from the yeast host cell, most of the leader peptide is processed from the fusion protein to yield a functional polypeptide (referred to herein as “PEP-1”) having the amino acid sequence of SEQ ID NO:2 (see boxed region in FIG. 1).

A typical Kunitz domain, e.g., that includes, SEQ ID NO:1, contains a number of invariant positions, e.g., positions corresponding to position 5, 14, 30, 33, 38, 45, 51 and 55 in the BPTI numbering scheme are cysteine. The spacing between these positions may vary to the extent allowable within the Kunitz domain fold, e.g., such that three disulfide bonds are formed. Other positions such as, for example, positions 6, 7, 8, 9, 20, 24, 25, 26, 27, 28, 29, 41, 42, 44, 46, 47, 48, 49, 50, 52, 53 and 54, or positions corresponding to those positions, can be any amino acid (including non-genetically encoded occurring amino acids). In a particularly preferred embodiment, one or more amino acids correspond to that of a native sequence (e.g., SEQ ID NO:32, see FIGS. 2A and 2B). In another embodiment, at least one variable position is different from that of the native sequence. In yet another preferred embodiment, the amino acids can each be individually or collectively substituted by a conservative or non-conservative amino acid substitution.

Conservative amino acid substitutions replace an amino acid with another amino acid of similar chemical nature and may have no affect on protein function. Non-conservative amino acid substitutions replace an amino acid with another amino acid of dissimilar chemical structure. Examples of conserved amino acid substitutions include, for example, Asn→Gln, Arg→Lys and Ser→Thr. In a preferred embodiment, 1, 2, 3, 4, 5, 6, 7, 8, 9, 10, 11, 12, 13, 14, 15, 16, 17, 18, 19, 20 and/or 21 of these amino acids can be independently or collectively, in any combination, selected to correspond to the corresponding position of SEQ ID NO:2.

Other positions, for example, positions 10, 11, 13, 15, 16, 17, 18, 19, 21, 22, 23, 31, 32, 34, 35, 39, 40, 43 and 45, or positions corresponding to those positions can be any of a selected set of amino acids. For example, SEQ ID NO:1 defines a set of possible sequences. Each member of this set contains, for example, a cysteine at positions 5, 14, 30, 51 and 55, and any one of a specific set of amino acids at positions 10, 11, 13, 15, 16, 17, 18, 19, 21, 22, 23, 31, 32, 34, 35, 39, 40, 43 and 45, or positions corresponding to those positions. In a preferred embodiment, 1, 2, 3, 4, 5, 6, 7, 8, 9, 10, 11, 12, 13, 14, 15, 16, 17, 18 and/or 19 of these amino acids can be independently or collectively, in any combination, selected to correspond to the corresponding position of SEQ ID NO:2. The polypeptide preferably has at least 80%, 85%, 90%, 95, 97, 98, or 99% identity to SEQ ID NO:2.

As used herein, the term “substantially identical” (or “substantially homologous”) is used herein to refer to a first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have similar activities. In the case of antibodies, the second antibody has the same specificity and has at least 50% of the affinity of the same.

Calculations of “homology” between two sequences can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent homology between two sequences can be accomplished using a mathematical algorithm. In a preferred embodiment, the percent homology between two amino acid sequences is determined using the Needleman and Wunsch (1970), J. Mol. Biol. 48:444-453, algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent homology between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a homology limitation) are a Blossum 62 scoring matrix with a gap penalty of 12, a gap extend penalty of 4, and a frameshift gap penalty of 5.

Useful polypeptides can also be encoded by a nucleic acid that hybridizes to a nucleic acid that encodes a polypeptide described herein. The nucleic acids can hybridize under medium, high, or very high stringency conditions. As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×SSC, 0.1% SDS at 65° C.; and (4) very high stringency hybridization conditions are 0.5M sodium phosphate, 7% SDS at 65° C., followed by one or more washes at 0.2×SSC, 1% SDS at 65° C.

### Modifications

It is possible to modify polypeptides that inhibit a Kunitz domain in a variety of ways. For example, the polypeptides can be attached to one or more polyethylene glycol moieties to stabilize the compound or prolong retention times, e.g., by at least 2, 4, 5, 8, 10, 15, 20, 50, 100, 500 or 1000 fold.

A polypeptide that inhibits kallikrein can be associated with (e.g., conjugated to) a polymer, e.g., a substantially non-antigenic polymers, such as polyalkylene oxides or polyethylene oxides. Suitable polymers will vary substantially by weight. Polymers having molecular number average weights ranging from about 200 to about 35,000 (or about 1,000 to about 15,000, and 2,000 to about 12,500) can be used. A plurality of polymer moieties can be attached to one polypeptide, e.g., at least two, three, or four such moieties, e.g., having an average molecular weight of about 2,000 to 7,000 Daltons.

For example, the polypeptide can be conjugated to a water soluble polymer, e.g., hydrophilic polyvinyl polymers, e.g. polyvinylalcohol and polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparan.

Other compounds can also be attached to the same polymer, e.g., a cytotoxin, a label, or another targeting agent or an unrelated agent. Mono-activated, alkoxy-terminated polyalkylene oxides (PAO's), e.g., monomethoxy-terminated polyethylene glycols (mPEG's); C₁₋₄ alkyl-terminated polymers; and bis-activated polyethylene oxides (glycols) can be used for crosslinking. See, e.g., U.S. Pat. No. 5,951,974.

### Methods

The kallikrein inhibitors described herein can be used in methods for the treatment of gout, particularly acute gout. As used herein, the term “treatment” refers to improvement of, reduction of the severity of, or stabilization of a symptom of gout. The method includes administering a non-naturally occurring inhibitor of kallikrein to a subject having, or suspected of having, gout.

In one embodiment, a method for treatment includes administration of a non-naturally occurring polypeptide comprising a Kunitz domain as the inhibitor of kallikrein. One embodiment of the method uses a polypeptide containing an amino acid sequence of SEQ ID NO:1 that has an affinity for kallikrein that is approximately 30-fold or more higher than that of a broad range serine protease, e.g., aprotinin, which is isolated from bovine lung and currently approved for use in coronary artery bypass grafting procedures (TRASYLOL™, Bayer Corporation Pharmaceutical Division, West Haven, Conn.).

Administration of the non-naturally occurring pKal inhibitor results in improvement of, a reduction in the severity of, or the stabilization of at least one symptom of gout or acute gout.

Symptoms of gout include pain, particularly arthralgia, and loss of joint function (e.g., range of motion, grip strength).

Gout symptoms may be measured using any appropriate technique or technology. For example, pain may be measured using a pain scale, such as a visual pain scale. Other useful measures include measures of joint function, such a measurements of range of motion, grip strength, and the like. Other measures that more generally account for overall function, such as measurements of the time the subject is able to maintain a standing position, or time to walk a specified distance are useful. Also useful are questionnaires and other measures of patient function, such as gout-related, arthritis-related, or general performance measures, such as the gout assessment questionnaire (GAQ), the health assessment questionnaire (HAQ), Katz index of activities of daily living (KIADL), or instrumental activities of daily living (IADL).

### Combination Therapy

The non-naturally occurring pKal inhibitor may be administered as part of a combination therapy for gout. Combination therapy involves the use of the non-naturally occurring pKal inhibitor along with an additional agent. The additional agent may be an additional acute gout therapeutic, an uric acid-lowering agent, a phagocytosis inhibitor, or a combination of two or more thereof.

Acute gout therapeutics include NSAIDs (e.g., indomethacin, acetaminophen, aspirin, naproxen sodium, ibuprofen, sulindac) and other analgesics (e.g., acetaminophen with codeine) and corticosteroids (e.g., prednisone, prednosolone, methylprednisolone) and corticosteroid-stimulating agents (e.g., corticotrophin), and anti-phagocytotic agents (e.g., colchicine).

Uric acid-lowering agents include xanthine oxidase (XO) inhibiting agents, uricosuric agents, and uric acid metabolizing agents. XO inhibitors useful in combination with a non-naturally occurring pKal inhibitor include, but are not limited to, allopurinol, oxypurinol, febuxostat ([2-[3-cyano-4-(2-methylpropoxyphenyl)-4-methylthiazole-5-carboxylic acid), Y-700 (1-[3-Cyano-4-(2,2-dimethylpropoxy)phenyl]-1H-pyrazole-4-carboxylic acid, and BOF-4272 ((±)-8-(3-methoxy-4-phenylsulphinylphenyl)pyrazolo[1,5-a]-1,3,5-triazine-4-(1H)-one).

Uricosuric agents include probenecid, sulfinpyrazone, benzbromarone, guaifenesin, salicylate, and losartan.

Phagocytosis inhibitors include colchicine.

The non-naturally occurring pKal inhibitor may be co-administered (e.g., administered as part of the same therapeutic composition or administered at the same time and by the same route, but as a separate composition) with the additional gout therapeutic. However, if the non-naturally occurring pKal inhibitor and the additional gout therapeutic are administered by different routes or on different dosing schedules, the non-naturally occurring pKal inhibitor and the additional gout therapeutic may be administered as separate compositions, and the pKal inhibitor may be administered before, concurrently with, or after the administration of the additional gout therapeutic. When the non-naturally occurring pKal inhibitor and the additional gout therapeutic are administered as separate compositions, the are considered to be administered as a “combination therapy” if the non-naturally occurring pKal inhibitor and the additional gout therapeutic are administered in a time frame that is overlapping (e.g., there are therapeutically effective amounts of the two agents present in the subject at the same time).

### Administration

The pKal inhibitor (alone or as part of a combination therapy) can be administered to a patient before, during, and/or after the onset of gout (e.g., an acute gout episode). The patient is generally a human, but may also be a non-human mammal. Human patients include adults, e.g., patients between ages 19-25, 26-40, 41-55, 56-75, and 76 and older, and pediatric patients, e.g., patients between ages 0-2, 3-6, 7-12, and 13-18.

The term “pharmaceutically acceptable” composition refers to a non-toxic carrier or excipient that may be administered to a patient, together with a pKal inhibitor described herein. The carrier or excipient is chosen to be compatible with the biological or pharmacological activity of the composition. The pKal inhibitors (and, in the case of combination therapy, additional gout therapeutics) described herein can be administered locally or systemically by any suitable means for delivery of an inhibitory amount of the inhibitor and/or additional gout therapeutic to a patient including but not limited to systemic administrations such as, for example, intravenous and inhalation. Parenteral administration is particularly preferred for the pKal inhibitor.

For parenteral administration, the pKal inhibitor and, as appropriate, the additional gout therapeutic, can be injected intravenously, intramuscularly, intraperitoneally, or subcutaneously. Subcutaneous injection and i.v. administration are preferred routes for parenteral administration. Also useful is local (intraarticular) injection, particularly when the involved joints are medium to large joints (e.g., hip, knee, elbow, ankle, wrist).

Typically, compositions for administration by injection are solutions in sterile isotonic aqueous buffer (e.g., sodium/potassium phosphate buffered saline). Other pharmaceutically acceptable carriers include, but are not limited to, sterile water, saline solution, and buffered saline (including buffers like phosphate or acetate), alcohol, vegetable oils, polyethylene glycols, gelatin, lactose, amylose, magnesium stearate, talc, silicic acid, paraffin, etc. Where necessary, the composition can also include a solubilizing agent and a local anesthetic such as lidocaine to ease pain at the site of the injection, preservatives, stabilizers, wetting agents, emulsifiers, salts, lubricants, etc. as long as they do not react deleteriously with the active compounds. Similarly, the composition can comprise conventional excipients, e.g., pharmaceutically acceptable organic or inorganic carrier substances suitable for parenteral, enteral or intranasal application which do not deleteriously react with the active compounds. Generally, the ingredients will be supplied either separately or mixed together in unit dosage form, for example, as a dry lyophilized powder or water free concentrate in a hermetically sealed container such as an ampoule, sachette, or vial indicating the quantity of active agent in activity units. Where the composition is to be administered by infusion, it can be dispensed with an infusion bottle containing sterile pharmaceutical grade “water for injection” or saline. Where the composition is to be administered by injection, a container (e.g., ampoule or vial) of sterile water for injection or saline can be provided so that the ingredients can be mixed prior to administration.

Exemplary formulations for subcutaneous administration of non-naturally occurring pKal inhibitors include buffered solutions containing a buffering agent (e.g., histidine or phosphate buffer) and a cryoprotectant (e.g., sucrose or sucrose and mannitol, optionally including a dextran such as dextran 40), and may be lyophilized for storage and distribution as described in U.S. Ser. No. 11/716,278, filed Mar. 9, 2007.

In one embodiment, the pKal inhibitor is administered to a patient as an intravenous infusion according to any approved procedure. In another embodiment, the pKal inhibitor is administered to a patient as a subcutaneous bolus. In another embodiment, the pKal inhibitor is administered to a patient by intraarticular injection into the affected joint(s). I.V. and intraarticular administration are typically carried out by a health care professional in a clinical setting (e.g., hospital, urgent care, or doctor's office), but subcutaneous injections may be self-administered or administered by a health care professional.

Parameters that can be evaluated for determining a dose of the kallikrein inhibitor for systemic administration, are described below with regards to DX-88 (a non-naturally occurring kallikrein inhibitor, SEQ ID NO:2). The total amount of circulating prekallikrein in plasma is reported to be approximately 500 nM to 600 nM (Silverberg et al., “The Contact System and Its Disorders,” in Blood: Principles and Practice of Hematology, Handin, R. et al., eds, J B Lippincott Co., Philadelphia, 1995). If all prekallikrein is activated, about 520 nmoles/L of DX-88 can be used to inhibit kallikrein in a stoichiometric manner. An individual having 5 L of plasma would require a dose of 2.6 micromoles DX-88, or approximately 18 mg based on the molecular weight of DX-88 of 7,054 Daltons. This was calculated as follows: the Kᵢ of DX88 is 0.044 nM. When it is desired to have a concentration of plasma kallikrein (PK) of, e.g., 1 nM, the formula Kᵢ=0.044 nM=[DX88]×[PK]/[DX88-PK]=[DX88]×1 nm/499 nM, indicates that the concentration of free DX-88 is 22.0 nM. Thus, the total amount of DX-88 needed would be 499+22 or 521 nM. The dose can be reduced proportionally if not all of the prekallikrein is activated or if a portion of the kallikrein is deactivated by an endogenous inhibitor, e.g., C1 esterase inhibitor (C1INH). Thus, in certain embodiments, about 5, 10, 15, 20, 30, 40, 60, 80, 120, 250, 500, 600, 700, 800, 1000 mg of DX-88 can be administered to a subject, in a single dose or in one or more doses spread over a twenty-four hour period. Consideration of several other factors may provide a more accurate estimation of the dose of DX-88 required in practice, such as patient age, weight, and severity of the gout symptoms.

In some embodiments, the kallikrein inhibitor polypeptide is administered in a dose of about 1-500 mg/m², preferably about 1-250 mg/m², 1-100 mg/m².

In combination therapies, the additional gout therapeutic(s) is administered at a dose suitable for the particular drug. For example, prednisone, a corticosteroid, is typically administered at 20-40 mg/day, with a tapered reduction in dosage following resolution of the acute gout episode. The NSAIDs indomethacin, ibuprofen, naproxen sodium, and sulindac are typically administered at 50 mg three times daily (tid), 600-800 mg tid, 825 as loading, dose followed by 275 mg tid, and 200 mg twice daily (bid), respectively. The anti-phagocytotic drug colchicine may be administered orally or by injection: for oral administration, a 1-1.2 mg loading dose is followed by 0.5 or 0.6 mg tablets taken hourly until (a) joint pain is relieved, (b) dose-limiting gastrointestinal side effects appear, or a total of 5 to 7 mg of colchicine has been administered; for parenteral administration, 1 mg is administered i.v. (to a maximum of four doses/day) until symptoms resolve or dose limiting toxicities (bone marrow suppression, renal damage, or altered liver function tests (LFTs)) appear. The uricosuric agents probenecid is typically given at 1 to 2 grams per day. Allopurinol is typically administered at 300 mg per day. Uricase (e.g., rasburicase) is administered at 4-24 mg per dose.

### Devices and Kits

Pharmaceutical compositions that include the kallikrein inhibitor (and optionally an additional gout therapeutic) can be administered with a medical device. The device can designed with features such as portability, room temperature storage, and ease of use so that it can be used in settings outside of a hospital or emergency room/urgent care facility (e.g., by the patient or a caregiver in the home or in a doctor's office). The device can include, e.g., one or more housings for storing pharmaceutical preparations that include a non-naturally occurring kallikrein inhibitor (and optionally an additional gout therapeutic), and can be configured to deliver one or more unit doses of the agent or agents.

I.V. administration may be by bolus or infusion, using appropriate injection or infusion devices (e.g., catheters, infusion pumps, implants, and the like). Subcutaneous injection may be as an infusion, for example using a catheter and infusion pump or implantable device. Many other devices, implants, delivery systems, and modules are also known.

When the pKal inhibitor (and optionally, an additional gout therapeutic) is distributed as a lyophilized powder, it must be reconstituted prior to use. Manual reconstitution (e.g., manual addition of diluent to the lyophilized formulation by injection through an injection port into the container containing the lyophilized formulation) may be used, or the pKal inhibitor (and optionally, an additional gout therapeutic) may be provided in a device configured for automatic reconstitution (e.g., automatic addition of the diluent to the lyophilized formulation), such as the BECTON-DICKINSON BD™ Liquid Dry Injector.

The non-naturally occurring kallikrein inhibitor can be provided in a kit. In one embodiment, the kit includes (a) a container that contains a composition that includes a non-naturally occurring kallikrein inhibitor, and (b) informational material that relates to the methods described herein and/or the use of the agents for therapeutic benefit. In an embodiment, the kit includes also includes an additional gout therapeutic. For example, the kit includes a first container that contains a composition that includes the non-naturally occurring kallikrein inhibitor, and a second container that includes the additional gout therapeutic.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the non-naturally occurring kallikrein inhibitor, e.g., in a suitable dose, dosage form, or mode of administration (e.g., a dose, dosage form, or mode of administration described herein), to treat a subject who has gout, such as acute gout. The information can be provided in a variety of formats, include printed text, computer readable material, video recording, or audio recording, or a information that provides a link or address to substantive material.

In addition to the non-naturally occurring kallikrein inhibitor (and, if present, the additional gout therapeutic), the composition in the kit can include other ingredients, such as a solvent or buffer, a stabilizer, or a preservative. The non-naturally occurring kallikrein inhibitor (and additional gout therapeutic, if present) can be provided in any form, e.g., liquid, dried or lyophilized form, preferably substantially pure and/or sterile. When the agents are provided in a liquid solution, the liquid solution preferably is an aqueous solution. When the agents are provided as a dried form, reconstitution generally is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the non-naturally occurring kallikrein inhibitor and an additional gout therapeutic, e.g., in a desired ratio. For example, the kit includes a plurality of syringes, ampoules, foil packets, blister packs, or medical devices, e.g., each containing a single combination unit dose. The containers of the kits can be air tight, waterproof (e.g., impermeable to changes in moisture or evaporation), and/or light-tight.

The kit optionally includes a device suitable for administration of the composition, e.g., a syringe or other suitable delivery device. The device can be provided pre-loaded with one or both of the agents or can be empty, but suitable for loading.

A number of embodiments of the invention have been described. Nevertheless, it will be understood that various modifications may be made without departing from the spirit and scope of the invention. Accordingly, other embodiments are within the scope of the following claims.

1. A method for treating acute gout, the method comprising administering an effective amount of a non-naturally occurring inhibitor of plasma kallikrein to an individual suffering from acute gout.

2. The method of claim 1, wherein the polypeptide comprises the amino acid sequence: Xaa1 Xaa2 Xaa3 Xaa4 Cys Xaa6 Xaa7 Xaa8 Xaa9 Xaa10 Xaa11 Gly Xaa13 Cys Xaa15 Xaa16 Xaa17 Xaa18 Xaa19 Xaa20 Xaa21 Xaa22 Xaa23 Xaa24 Xaa25 Xaa26 Xaa27 Xaa28 Xaa29 Cys Xaa31 Xaa32 Phe Xaa34 Xaa35 Gly Gly Cys Xaa39 Xaa40 Xaa41 Xaa42 Xaa43 Xaa44 Xaa45 Xaa46 Xaa47 Xaa48 Xaa49 Xaa50 Cys Xaa52 Xaa53 Xaa54 Cys Xaa56 Xaa57 Xaa58 (SEQ ID NO:1),
wherein Xaa1, Xaa2, Xaa3, Xaa4, Xaa56, Xaa57 or Xaa58 are each individually an amino acid or absent;
Xaa10 is an amino acid selected from the group consisting of: Asp and Glu;
Xaa11 is an amino acid selected from the group consisting of: Asp, Gly, Ser, Val, Asn, Ile, Ala and Thr;
Xaa13 is an amino acid selected from the group consisting of: Arg, His, Pro, Asn, Ser, Thr, Ala, Gly, Lys and Gln;
Xaa15 is an amino acid selected from the group consisting of: Arg, Lys, Ala, Ser, Gly, Met, Asn and Gln;
Xaa16 is an amino acid selected from the group consisting of: Ala, Gly, Ser, Asp and Asn;
Xaa17 is an amino acid selected from the group consisting of: Ala, Asn, Ser, Ile, Gly, Val, Gln and Thr;
Xaa18 is an amino acid selected from the group consisting of: His, Leu, Gln and Ala;
Xaa19 is an amino acid selected from the group consisting of: Pro, Gln, Leu, Asn and Ile;
Xaa21 is an amino acid selected from the group consisting of: Trp, Phe, Tyr, His and Ile;
Xaa22 is an amino acid selected from the group consisting of: Tyr and Phe;
Xaa23 is an amino acid selected from the group consisting of: Tyr and Phe;
Xaa31 is an amino acid selected from the group consisting of: Glu, Asp, Gln, Asn, Ser, Ala, Val, Leu, Ile and Thr;
Xaa32 is an amino acid selected from the group consisting of: Glu, Gln, Asp Asn, Pro, Thr, Leu, Ser, Ala, Gly and Val;
Xaa34 is an amino acid selected from the group consisting of: Thr, Ile, Ser, Val, Ala, Asn, Gly and Leu;
Xaa35 is an amino acid selected from the group consisting of: Tyr, Trp and Phe;
Xaa39 is an amino acid selected from the group consisting of: Glu, Gly, Ala, Ser and Asp;
Xaa40 is an amino acid selected from the group consisting of: Gly and Ala;
Xaa43 is an amino acid selected from the group consisting of: Asn and Gly;
Xaa45 is an amino acid selected from the group consisting of: Phe and Tyr; and wherein the polypeptide inhibits kallikrein.

3. The method of claim 2, wherein Xaa10 is Asp.

4. The method of claim 2, wherein Xaa11 is Asp.

5. The method of claim 2, wherein Xaa13 is Pro, Xaa15 is Arg, Xaa16 is Ala, Xaa17 is Ala, Xaa18 is His and Xaa19 is Pro.

6. The method of claim 2, wherein Xaa21 is Trp.

7. The method of claim 2, wherein Xaa31 is Glu.

8. The method of claim 2, wherein Xaa32 is Glu.

9. The method of claim 2, wherein Xaa34 is Ile.

10. The method of claim 2, wherein Xaa35 is Tyr.

11. The method of claim 2, wherein Xaa39 is Glu.

12. The method of claim 2, wherein the polypeptide comprises: Met His Ser Phe Cys Ala Phe Lys Ala Asp Asp Gly Pro Cys Arg Ala Ala His Pro Arg Trp Phe Phe Asn Ile Phe Thr Arg Gln Cys Glu Glu Phe Ile Tyr Gly Gly Cys Glu Gly Asn Gln Asn Arg Phe Glu Ser Leu Glu Glu Cys Lys Lys Met Cys Thr Arg Asp (amino acids 3-60 of SEQ ID NO:2).

13. The method of claim 12, wherein the polypeptide further comprises a Glu-Ala sequence prior to the amino terminal Met residue.

14. The method of claim 1, further comprising administering an additional gout therapeutic to said individual.

15. The method of claim 14, wherein the additional gout therapeutic is selected from the group consisting of: non-steroidal anti-inflammatory drugs (NSAIDs), corticosteroids, analgesics, phagocytosis inhibiting agents, xanthine oxidase inhibiting agents, uricosuric agents, and uric acid metabolizing agents.

16. The method of claim 15, wherein the additional gout therapeutic is an NSAID.

17. The method of claim 15, wherein the additional gout therapeutic is a corticosteroid.

18. The method of claim 15, wherein the additional gout therapeutic is a phagocytosis inhibiting agent.

19. A kit, comprising:
a container comprising a non-naturally occurring plasma kallikrein inhibitor; and
instructions for use of said kallikrein inhibitor for the treatment of gout.


SEQUENCE LISTING (58 sequences)

SEQ ID NO: 1 - protein, 58 aa, Artificial Sequence
  Description of Artificial Sequence: Synthetic polypeptide
  MOD_RES (1)..(4): Any amino acid or not present
  MOD_RES (6)..(9): Any amino acid
  MOD_RES (10)..(10): Asp or Glu
  MOD_RES (11)..(11): Asp, Gly, Ser, Val, Asn, Ile, Ala or Thr
  MOD_RES (13)..(13): Arg, His, Pro, Asn, Ser, Thr, Ala, Gly, Lys or Gln
  MOD_RES (15)..(15): Arg, Lys, Ala, Ser, Gly, Met, Asn or Gln
  MOD_RES (16)..(16): Ala, Gly, Ser, Asp or Asn
  MOD_RES (17)..(17): Ala, Asn, Ser, Ile, Gly, Val, Gln or Thr
  MOD_RES (18)..(18): His, Leu, Gln or Ala
  MOD_RES (19)..(19): Pro, Gln, Leu, Asn or Ile
  MOD_RES (20)..(20): Any amino acid
  MOD_RES (21)..(21): Trp, Phe, Tyr, His or Ile
  MOD_RES (22)..(23): Tyr or Phe
  MOD_RES (24)..(29): Any amino acid
  MOD_RES (31)..(31): Glu, Asp, Gln, Asn, Ser, Ala, Val, Leu, Ile or Thr
  MOD_RES (32)..(32): Glu, Gln, Asp, Asn, Pro, Thr, Leu, Ser, Ala, Gly or Val
  MOD_RES (34)..(34): Thr, Ile, Ser, Val, Ala, Asn, Gly or Leu
  MOD_RES (35)..(35): Tyr, Trp or Phe
  MOD_RES (39)..(39): Glu, Gly, Ala, Ser or Asp
  MOD_RES (40)..(40): Gly or Ala
  MOD_RES (41)..(42): Any amino acid
  MOD_RES (43)..(43): Asn or Gly
  MOD_RES (44)..(44): Any amino acid
  MOD_RES (45)..(45): Phe or Tyr
  MOD_RES (46)..(50): Any amino acid
  MOD_RES (52)..(54): Any amino acid
  MOD_RES (56)..(58): Any amino acid or not present
  See specification as filed for detailed description of substitutions and preferred embodiments
XXXXCXXXXXXGXCXXXXXXXXXXXXXXXCXXFXXGGCXXXXXXXXXXXXCXXXCXXX

SEQ ID NO: 2 - protein, 60 aa, Artificial Sequence
  Description of Artificial Sequence: Synthetic polypeptide
EAMHSFCAFKADDGPCRAAHPRWFFNIFTRQCEEFIYGGCEGNQNRFESLEECKKMCTRD

SEQ ID NO: 3 - DNA, 180 bp, Artificial Sequence
  Description of Artificial Sequence: Synthetic polynucleotide
  CDS (1)..(180)
gaggctatgcactctttctgtgctttcaaggctgacgacggtccgtgcagagctgctcacccaagatggttcttcaacatcttcacgcgtcaatgcgaggagttcatctacggtggttgtgagggtaaccaaaacagattcgagtctctagaggagtgtaagaagatgtgtactagagac

SEQ ID NO: 4 - protein, 58 aa, Artificial Sequence
  Description of Artificial Sequence: Synthetic polypeptide
MHSFCAFKADDGPCKANHLRFFFNIFTRQCEEFSYGGCGGNQNRFESLEECKKMCTRD

SEQ ID NO: 5 - protein, 58 aa, Artificial Sequence
  Description of Artificial Sequence: Synthetic polypeptide
MHSFCAFKADDGHCKANHQRFFFNIFTRQCEEFTYGGCGGNQNRFESLEECKKMCTRD

SEQ ID NO: 6 - protein, 58 aa, Artificial Sequence
  Description of Artificial Sequence: Synthetic polypeptide
MHSFCAFKADDGHCKANHQRFFFNIFTRQCEQFTYGGCAGNQNRFESLEECKKMCTRD

SEQ ID NO: 7 - protein, 58 aa, Artificial Sequence
  Description of Artificial Sequence: Synthetic polypeptide
MHSFCAFKADDGHCKASLPRFFFNIFTRQCEEFIYGGCGGNQNRFESLEECKKMCTRD

SEQ ID NO: 8 - protein, 58 aa, Artificial Sequence
  Description of Artificial Sequence: Synthetic polypeptide
MHSFCAFKADDGHCKANHQRFFFNIFTRQCEEFSYGGCGGNQNRFESLEECKKMCTRD

SEQ ID NO: 9 - protein, 58 aa, Artificial Sequence
  Description of Artificial Sequence: Synthetic polypeptide
MHSFCAFKADDGHCKGAHLRFFFNIFTRQCEEFIYGGCEGNQNRFESLEECKKMCTRD

SEQ ID NO: 10 - protein, 58 aa, Artificial Sequence
  Description of Artificial Sequence: Synthetic polypeptide
MHSFCAFKADDGRCKGAHLRFFFNIFTRQCEEFIYGGCEGNQNRFESLEECKKMCTRD

SEQ ID NO: 11 - protein, 58 aa, Artificial Sequence
  Description of Artificial Sequence: Synthetic polypeptide
MHSFCAFKADGGRCRGAHPRWFFNIFTRQCEEFSYGGCGGNQNRFESLEECKKMCTRD

SEQ ID NO: 12 - protein, 58 aa, Artificial Sequence
  Description of Artificial Sequence: Synthetic polypeptide
MHSFCAFKADDGPCRAAHPRWFFNIFTRQCEEFSYGGCGGNQNRFESLEECKKMCTRD

SEQ ID NO: 13 - protein, 58 aa, Artificial Sequence
  Description of Artificial Sequence: Synthetic polypeptide
MHSFCAFKADVGRCRGAHPRWFFNIFTRQCEEFSYGGCGGNQNRFESLEECKKMCTRD

SEQ ID NO: 14 - protein, 58 aa, Artificial Sequence
  Description of Artificial Sequence: Synthetic polypeptide
MHSFCAFKADVGRCRGAQPRFFFNIFTRQCEEFSYGGCGGNQNRFESLEECKKMCTRD

SEQ ID NO: 15 - protein, 58 aa, Artificial Sequence
  Description of Artificial Sequence: Synthetic polypeptide
MHSFCAFKADDGSCRAAHLRWFFNIFTRQCEEFSYGGCGGNQNRFESLEECKKMCTRD

SEQ ID NO: 16 - protein, 58 aa, Artificial Sequence
  Description of Artificial Sequence: Synthetic polypeptide
MHSFCAFKAEGGSCRAAHQRWFFNIFTRQCEEFSYGGCGGNQNRFESLEECKKMCTRD

SEQ ID NO: 17 - protein, 58 aa, Artificial Sequence
  Description of Artificial Sequence: Synthetic polypeptide
MHSFCAFKADDGPCRGAHLRFFFNIFTRQCEEFSYGGCGGNQNRFESLEECKKMCTRD

SEQ ID NO: 18 - protein, 58 aa, Artificial Sequence
  Description of Artificial Sequence: Synthetic polypeptide
MHSFCAFKADDGHCRGALPRWFFNIFTRQCEEFSYGGCGGNQNRFESLEECKKMCTRD

SEQ ID NO: 19 - protein, 58 aa, Artificial Sequence
  Description of Artificial Sequence: Synthetic polypeptide
MHSFCAFKADSGNCRGNLPRFFFNIFTRQCEEFSYGGCGGNQNRFESLEECKKMCTRD

SEQ ID NO: 20 - protein, 58 aa, Artificial Sequence
  Description of Artificial Sequence: Synthetic polypeptide
MHSFCAFKADSGRCRGNHQRFFFNIFTRQCEEFSYGGCGGNQNRFESLEECKKMCTRD

SEQ ID NO: 21 - protein, 58 aa, Artificial Sequence
  Description of Artificial Sequence: Synthetic polypeptide
MHSFCAFKADGGRCRAIQPRWFFNIFTRQCEEFSYGGCGGNQNRFESLEECKKMCTRD

SEQ ID NO: 22 - protein, 58 aa, Artificial Sequence
  Description of Artificial Sequence: Synthetic polypeptide
MHSFCAFKADDGRCRGAHPRWFFNIFTRQCEEFSYGGCGGNQNRFESLEECKKMCTRD

SEQ ID NO: 23-26 - skipped (4 entries)

SEQ ID NO: 27 - DNA, 548 bp, Artificial Sequence
  Description of Artificial Sequence: Synthetic polynucleotide
  CDS (54)..(488)
cgacttttaacgacaacttgagaagatcaaaaaacaactaattattcgaaacgatgagattcccatctatcttcactgctgttttgttcgctgcttcctctgctttggctgctccagttaacaccactactgaagacgagactgctcaaattcctgctgaggctgtcatcggttactctgacttggaaggtgacttcgacgtcgctgttttgccattctctaactctactaacaacggtttgttgttcatcaacactaccatcgcttctatcgctgctaaggaggaaggtgtttccctcgagaagagagaggctatgcactctttctgtgctttcaaggctgacgacggtccgtgcagagctgctcacccaagatggttcttcaacatcttcacgcgtcaatgcgaggagttcatctacggtggttgtgagggtaaccaaaacagattcgagtctctagaggagtgtaagaagatgtgtactagagactagtaagaattcgccttagacatgactgttcctcagttcaagttgggcacttacgagaag

SEQ ID NO: 28 - skipped

SEQ ID NO: 29 - protein, 58 aa, Bos taurus [sequence omitted]

SEQ ID NO: 30 - protein, 58 aa, Artificial Sequence
  Description of Artificial Sequence: Synthetic polypeptide
KEDSCQLGYSAGPCMGMTSRYFYNGTSMACETFQYGGCMGNGNNFVTEKECLQTCRTV

SEQ ID NO: 31 - protein, 58 aa, Artificial Sequence
  Description of Artificial Sequence: Synthetic polypeptide
TVAACNLPIVRGPCRAFIQLWAFDAVKGKCVLFPYGGCQGNGNKFYSEKECREYCGVP

SEQ ID NO: 32 - protein, 58 aa, Homo sapiens
MHSFCAFKADDGPCKAIMKRFFFNIFTRQCEEFIYGGCEGNQNRFESLEECKKMCTRD

SEQ ID NO: 33 - protein, 58 aa, Artificial Sequence
  Description of Artificial Sequence: Synthetic polypeptide
KPDFCFLEEDPGICRGYITRYFYNNQTKQCERFKYGGCLGNMNNFETLEECKNICEDG

SEQ ID NO: 34 - protein, 58 aa, Artificial Sequence
  Description of Artificial Sequence: Synthetic polypeptide
GPSWCLTPADRGLCRANENRFYYNSVIGKCRPFKYSGCGGNENNFTSKQECLRACKKG

SEQ ID NO: 35 - protein, 58 aa, Artificial Sequence
  Description of Artificial Sequence: Synthetic polypeptide
LPNVCAFPMEKGPCQTYMTRWFFNFETGECELFAYGGCGGNSNNFLRKEKCEKFCKFT

SEQ ID NO: 36 - protein, 58 aa, Artificial Sequence
  Description of Artificial Sequence: Synthetic polypeptide
ETDICKLPKDEGTCRDFILKWYYDPNTKSCARFWYGGCGGNENKFGSQKECEKVCAPV

SEQ ID NO: 37 - protein, 58 aa, Artificial Sequence
  Description of Artificial Sequence: Synthetic polypeptide
NAEICLLPLDYGPCRALLLRYYYDRYTQSCRQFLYGGCEGNANNFYTWEACDDACWRI

SEQ ID NO: 38 - protein, 61 aa, Artificial Sequence
  Description of Artificial Sequence: Synthetic polypeptide
VPKVCRLQVSVDDQCEGSTEKYFFNLSSMTCEKFFSGGCHRNRIENRFPDEATCMGFCAPK

SEQ ID NO: 39 - protein, 58 aa, Artificial Sequence
  Description of Artificial Sequence: Synthetic polypeptide
IPSFCYSPKDEGLCSANVTRYYFNPRYRTCDAFTYTGCGGNDNNFVSREDCKRACAKA

SEQ ID NO: 40 - protein, 59 aa, Artificial Sequence
  Description of Artificial Sequence: Synthetic polypeptide
RNREVCSEQAETGPCRAMISRWYFDVTEGKCAPFFYGGCGGNRNNFDTEEYCMAVCGSA

SEQ ID NO: 41 - protein, 58 aa, Artificial Sequence
  Description of Artificial Sequence: Synthetic polypeptide
RPDFCLEPPYTGPCVAMFPRYFYNAKAGLCQTFVYGGCMGNGNNFKSAEDCMRTCGGA

SEQ ID NO: 42 - protein, 58 aa, Artificial Sequence
  Description of Artificial Sequence: Synthetic polypeptide
RPDFCQLGYSAGPCVAMFPRYFYNGTSMACQTFVYGGCMGNGNNFVTEKDCLQTCRGA

SEQ ID NO: 43 - protein, 58 aa, Artificial Sequence
  Description of Artificial Sequence: Synthetic polypeptide
RPDFCQLGYSAGPCVAMFPRYFYNGASMACQTFVYGGCMGNGNNFVTEKDCLQTCRGA

SEQ ID NO: 44 - protein, 58 aa, Artificial Sequence
  Description of Artificial Sequence: Synthetic polypeptide
RPDFCQLGYSAGPCVAMFPRYFYNGTSMACETFVYGGCMGNGNNFVTEKDCLQTCRGA

SEQ ID NO: 45 - protein, 58 aa, Artificial Sequence
  Description of Artificial Sequence: Synthetic polypeptide
RPDFCQLGYSAGPCVGMFSRYFYNGTSMACQTFVYGGCMGNGNNFVTEKDCLQTCRGA

SEQ ID NO: 46 - protein, 62 aa, Artificial Sequence
  Description of Artificial Sequence: Synthetic polypeptide
EAEARPDFCLEPPYTGPCIAFFPRYFYNAKAGLCQTFVYGGCMGNGNNFKSAEDCMRTCGGA

SEQ ID NO: 47 - protein, 56 aa, Artificial Sequence
  Description of Artificial Sequence: Synthetic polypeptide
AACNLPIVRGPCIAFFPRWAFDAVKGKCVLFPYGGCQGNGNKFYSEKECREYCGVP

SEQ ID NO: 48 - protein, 56 aa, Artificial Sequence; identical to SEQ ID NO: 47
  Description of Artificial Sequence: Synthetic polypeptide

SEQ ID NO: 49 - protein, 56 aa, Artificial Sequence
  Description of Artificial Sequence: Synthetic polypeptide
EACNLPIVRGPCIAFFPRWAFDAVKGKCVLFPYGGCQGNGNKFYSEKECREYCGVP

SEQ ID NO: 50 - protein, 60 aa, Artificial Sequence
  Description of Artificial Sequence: Synthetic polypeptide
EAVREVCSEQAETGPCIAFFPRWYFDVTEGKCAPFFYGGCGGNRNNFDTEEYCMAVCGSA

SEQ ID NO: 51 - protein, 60 aa, Artificial Sequence
  Description of Artificial Sequence: Synthetic polypeptide
EANAEICLLPLDYGPCIAFFPRYYYDRYTQSCRQFLYGGCEGNANNFYTWEACDDACWRI

SEQ ID NO: 52 - protein, 60 aa, Artificial Sequence
  Description of Artificial Sequence: Synthetic polypeptide
EAKPDFCFLEEDPGICIGFFPRYFYNNQAKQCERFVYGGCLGNMNNFETLEECKNICEDG

SEQ ID NO: 53 - protein, 60 aa, Artificial Sequence
  Description of Artificial Sequence: Synthetic polypeptide
EAETDICKLPKDEGTCIAFFPRWYYDPNTKSCARFVYGGCGGNENKFGSQKECEKVCAPV

SEQ ID NO: 54 - protein, 304 aa, Homo sapiens
MIYTMKKVHALWASVCLLLNLAPAPLNADSEEDEEHTIITDTELPPLKLMHSFCAFKADDGPCKAIMKRFFFNIFTRQCEEFIYGGCEGNQNRFESLEECKKMCTRDNANRIIKTTLQQEKPDFCFLEEDPGICRGYITRYFYNNQTKQCERFKYGGCLGNMNNFETLEECKNICEDGPNGFQVDNYGTQLNAVNNSLTPQSTKVPSLFEFHGPSWCLTPADRGLCRANENRFYYNSVIGKCRPFKYSGCGGNENNFTSKQECLRACKKGFIQRISKGGLIKTKRKRKKQRVKIAYEEIFVKNM

SEQ ID NO: 55 - protein, 58 aa, Bos taurus [sequence omitted]

SEQ ID NO: 56 - protein, 145 aa, Artificial Sequence
  Description of Artificial Sequence: Synthetic polypeptide
MRFPSIFTAVLFAASSALAAPVNTTTEDETAQIPAEAVIGYSDLEGDFDVAVLPFSNSTNNGLLFINTTIASIAAKEEGVSLEKREAMHSFCAFKADDGPCRAAHPRWFFNIFTRQCEEFIYGGCEGNQNRFESLEECKKMCTRD

SEQ ID NO: 57 - protein, 58 aa, Artificial Sequence; identical to SEQ ID NO: 1
  Description of Artificial Sequence: Synthetic polypeptide
  MOD_RES (1)..(4): Any amino acid except Cys
  MOD_RES (6)..(11): Any amino acid except Cys
  MOD_RES (13)..(13): Any amino acid except Cys
  MOD_RES (15)..(29): Any amino acid except Cys
  MOD_RES (31)..(32): Any amino acid except Cys
  MOD_RES (34)..(35): Any amino acid except Cys
  MOD_RES (39)..(50): Any amino acid except Cys
  MOD_RES (52)..(54): Any amino acid except Cys
  MOD_RES (56)..(58): Any amino acid except Cys
  See specification as filed for detailed description of substitutions and preferred embodiments

SEQ ID NO: 58 - protein, 58 aa, Artificial Sequence
  Description of Artificial Sequence: Synthetic polypeptide
  MOD_RES (10)..(11): Any amino acid
  MOD_RES (13)..(13): Any amino acid
  MOD_RES (15)..(19): Any amino acid
  MOD_RES (21)..(21): Any amino acid
  MOD_RES (31)..(32): Any amino acid
  MOD_RES (34)..(35): Any amino acid
  MOD_RES (39)..(39): Any amino acid
  See specification as filed for detailed description of substitutions and preferred embodiments
MHSFCAFKAXXGXCXXXXXRXFFNIFTRQCXXFXXGGCXGNQNRFESLEECKKMCTRD